(19) United States
(12) Patent Application Publication
(10) Pub. No.: US 2018/0014813 A1
(43) Pub. Date: Jan. 18, 2018

(54) ULTRASOUND TRANSDUCER PROBE WITH HEAT TRANSFER DEVICE

(71) Applicant: EchoNous, Inc., Redmond, WA (US)

(72) Inventors: Todd Willsie, Seattle, WA (US); Greg Nieminen, Bothell, WA (US)

(21) Appl. No.: 15/650,409

(22) Filed: Jul. 14, 2017

Related U.S. Application Data
(60) Provisional application No. 62/363,160, filed on Jul. 15, 2016.

(51) Int. Cl. A61B 8/00; G01S 15/02; G01S 15/89

(52) U.S. Cl. CPC A61B 8/4483; G01S 15/8911; A61B 8/4245; A61B 8/4455; G01S 15/02

(57) ABSTRACT

Ultrasound systems, devices, and methods for removing heat from within an ultrasound transducer probe are provided herein. An ultrasound transducer probe may include an imaging surface having one or more transducer elements, electronic circuitry, a heat exchanger and a housing. The housing at least partially surrounds the imaging surface, electronic circuitry and heat exchanger or the heat exchanger may simply be operatively coupled to the probe. The electronic circuitry may include processing circuitry that controls transmission of an ultrasound signal from the one or more transducer elements, and driving circuitry operatively coupled to the one or more transducer elements and the processing circuitry. The driving circuitry is configured to drive the transmission of the ultrasound signal by the one or more transducer elements in response to a control signal received from the processing circuitry. The heat exchanger includes a conduit for containing a flow of cooling fluid.

### BACKGROUND

### Technical Field

The present application pertains to ultrasound systems, and more particularly to ultrasound systems including a heat transfer device.

### Description of the Related Art

Ultrasound imaging is a useful imaging modality in a number of environments. For example, in the field of healthcare, internal structures of a patient's body may be imaged before, during or after a therapeutic intervention. A healthcare professional may hold a portable ultrasound probe, or transducer, in proximity to the patient and move the transducer as appropriate to visualize one or more target structures in a region of interest in the patient. A transducer may be placed on the surface of the body or, in some procedures, a transducer is inserted inside the patient's body. The healthcare professional coordinates the movement of the transducer so as to obtain a desired representation on a screen, such as a two-dimensional cross-section of a three-dimensional volume.

Ultrasound may also be used to measure functional aspects of a patient, such as organ movement and blood flow in the patient. Doppler measurements, for example, are effective in measuring the direction and speed of movement of a structure, such as a heart valve or blood cells flowing in a vessel, relative to the transducer. Doppler echocardiography is widely used for evaluating the cardiocirculatory system of patients with known or suspected cardiovascular disease.

For many years, ultrasound imaging was effectively confined to large equipment operating in a hospital environment. Recent technological advances, however, have produced smaller ultrasound systems that increasingly are deployed in frontline point-of-care environments, e.g., doctor's offices. Nevertheless, smaller ultrasound systems typically lack the power, thermal management, and processing capabilities of larger systems. This generally results in limited runtime of the ultrasound imaging components, lower image resolution, and fewer features or modes of operation.

In conventional ultrasound imaging systems, much or most of the electronic circuitry or components that generate heat are located within equipment connected to the ultrasound probe by one or more cables. This allows some or most of the heat generating components to be physically separate from the transducer probe, and thus maintaining the surfaces of the probe at a safe temperature during operational use is relatively straightforward.

However, when designing smaller ultrasound systems (e.g., systems having a handheld computing device, such as a tablet, in place of equipment traditionally located on an equipment cart), some of the heat-generating components which were traditionally positioned within the equipment cart separate from the transducer probe may be positioned instead at least partially within the transducer probe itself, causing additional heat to be generated within the probe. This may result in the probe surface temperature being increased to an uncomfortable or unsafe level.

### BRIEF SUMMARY

The present disclosure, in part, addresses a desire for smaller ultrasound systems, having greater portability, lower cost, and ease of use for different modes of ultrasound imaging, while at the same time providing high quality measurements and effective thermal management.

Embodiments provided by the present disclosure reduce an amount of heat within an ultrasound transducer probe, and/or reduce the temperature at an outer surface of the probe. Electronic components and circuitry within the probe (e.g., driving circuitry, processing circuitry, transducer, and the like) generate heat, which, without a heat transfer or cooling system, is transferred by convection and/or radiation to an outer surface of the probe. The outer surface of the probe may thus rise in temperature to an unsafe, impermissible or otherwise undesirable level. Because ultrasound transducer probes are typically sealed (which may be required by applicable laws or regulations), forced convection inside of the sealed transducer is generally impractical and/or ineffective.

The performance of portable ultrasound devices may thus be limited by the temperature of the outer surface of the probe and/or by the amount of heat generated within the probe. By reducing the heat within the probe, and thereby reducing the temperatures experienced at the outer surfaces of the probe, embodiments provided herein provide significant benefits over conventional ultrasound devices and systems. For example, reducing the heat within the probe allows for operating the ultrasound device for a longer period of time, while staying within regulatory limits with respect to the temperature of the ultrasound transducer probe during patient contact. Additionally, by reducing the heat within the probe, heat-generating electronic components and circuitry which were traditionally confined to being positioned within external equipment may be moved into the probe without resulting in unsafe operating temperatures, thereby facilitating further miniaturization of such systems.

In at least one embodiment, an ultrasound transducer probe is provided that includes an imaging surface having one or more transducer elements, electronic circuitry, a heat exchanger and a housing. The housing at least partially surrounds the imaging surface, electronic circuitry and heat exchanger. The electronic circuitry may include processing circuitry that controls transmission of an ultrasound signal from the one or more transducer elements, and driving circuitry operatively coupled to the one or more transducer elements and the processing circuitry. The driving circuitry drives the transmission of the ultrasound signal by the one or more transducer elements in response to a control signal received from the processing circuitry. The heat exchanger includes a conduit for containing a flow of cooling fluid.

The ultrasound transducer probe may be coupled to a cable in a sealed or fluid-tight fashion. The cable may include portions of the conduit (e.g., an inlet tube and an outlet tube) that are mated to inlet and outlet ports, respectively, of the heat exchanger. The cable may further include electrical wires for transmitting signals between the probe and a computing device. A fluid pump, such as a miniature air blower or fluid circulator, may be included within the computing device (or alternatively within or operatively coupled to the conduit at any position along the cable or the transducer probe), and pumps a cooling fluid (e.g., ambient air) through the conduit. The fluid thus enters the heat exchanger, where it is heated by absorbing heat from within the probe, and exits the heat exchanger and the probe. The heated air may then be vented to an outside environment.

In another embodiment, an ultrasound system is provided that includes a transducer probe. The transducer probe includes an imaging surface including one or more transducer elements, electronic circuitry coupled to the one or more transducer elements, the electronic circuitry operatively generating heat, and a heat exchanger including a conduit configured to convey a flow of fluid. The heat exchanger is configured to absorb at least a portion of the heat generated by the electronic circuitry into the flow of fluid. The transducer probe further includes a housing at least partially surrounding the electronic circuitry, the heat exchanger and the imaging surface. The ultrasound system further includes a computing device operatively coupled to the transducer probe.

In yet another embodiment, a method for removing heat from within an ultrasound transducer probe is provided that includes providing a heat exchanger preferably within the ultrasound transducer probe, the heat exchanger including a heat exchanger conduit configured to convey a flow of fluid, the heat exchanger conduit including an inlet and an outlet; coupling an inlet conduit to the inlet of the heat exchanger conduit; coupling an outlet conduit to the outlet of the heat exchanger conduit; and coupling a fluid pump to the inlet conduit, the fluid pump being configured to provide the flow of fluid by pumping a cooling fluid into the inlet conduit.

### BRIEF DESCRIPTION OF THE SEVERAL VIEWS OF THE DRAWINGS

FIG. 1 is a schematic illustration of an ultrasound imaging device, in accordance with one or more embodiments of the present disclosure.

FIG. 2 is a cutaway view showing internal components of a transducer probe included in the ultrasound imaging device of FIG. 1, in accordance with one or more embodiments.

### DETAILED DESCRIPTION

A transducer probe in an ultrasound imaging device may include a heat transfer device operatively coupled to the transducer probe for removing heat from within the probe, and thereby reducing an operational temperature of the outer surfaces of the probe (e.g., the imaging surface, or an outer surface of the probe housing). The heat transfer device may include, for example, a heat exchanger that houses a conduit. The conduit may receive a flow of a cooling fluid, such as air, that absorbs heat from within the probe and vents the heat, e.g., through one or more vents outside of and away from the probe.

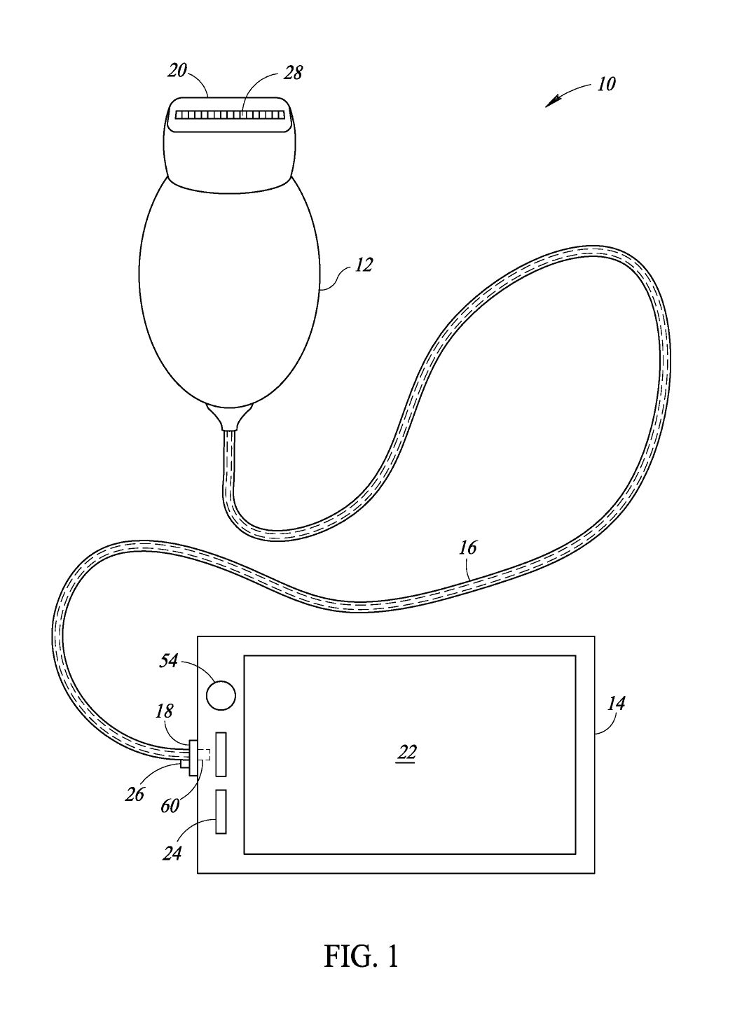

FIG. 1 is a schematic illustration of an ultrasound imaging device 10 (referred to herein as “ultrasound device” 10), in accordance with one or more embodiments of the present disclosure. The ultrasound device 10 includes an ultrasound transducer probe 12 that is electrically coupled to a computing device 14 by a cable 16. The cable 16 includes a connector 18 that detachably connects the probe 12 to the computing device 14. As shown in FIG. 1, the ultrasound device 10 may be a portable ultrasound device, i.e., the probe 12 may be a handheld probe that is connected to a portable computing device 14, such as a tablet computer, laptop, a hand-held device, or the like.

The probe 12 is configured to transmit an ultrasound signal toward a target structure in a region of interest. The probe 12 is further configured to receive echo signals returning from the target structure in response to transmission of the ultrasound signal. To that end, the probe 12 includes an imaging surface 20 that includes one or more transducer elements 28 that are capable of transmitting an ultrasound signal and receiving subsequent echo signals. In various embodiments, the transducer elements 28 may be arranged as elements of a phased array transducer. Suitable phased array transducers are known in the field of ultrasound technology.

The ultrasound device 10 further includes processing circuitry and driving circuitry. In part, the processing circuitry controls the transmission of the ultrasound signal from the transducer elements 28. The driving circuitry is operatively coupled to the transducer elements 28 for driving the transmission of the ultrasound signal. The driving circuitry may drive the transmission of the ultrasound signal in response to a control signal received from the processing circuitry. In one or more embodiments, the processing circuitry and the driving circuitry are located within the probe 12.

The ultrasound device 10 also includes a power supply that provides power to the driving circuitry for transmission of the ultrasound signal, for example, in a pulsed wave or a continuous wave mode of operation. In one or more embodiments, the power supply may be located within the probe 12. Additionally, or alternatively, the ultrasound device 10 may include a power supply located within the computing device 14 and configured to supply power to one or more electronic components located within the probe 12, e.g., via the cable 16.

The computing device 14 shown in FIG. 1 includes a display screen 22 and a user interface 24. The display screen 22 may use any type of display technology including, but not limited to, LED display technology. The display screen 22 is used to display one or more images generated from echo data obtained from the echo signals received in response to transmission of an ultrasound signal. In some embodiments, the display screen 22 may be a touch screen capable of receiving input from a user that touches the screen. In some embodiments, the user interface 24 may include one or more buttons, knobs, switches, and the like, capable of receiving input from a user of the ultrasound device 10.

The computing device 14 may further include one or more audio speakers 54 that may be used to generate audible representations of echo signals or other features derived from operation of the ultrasound device 10.

The cable 16 includes electrical wires for transmitting and receiving electrical signals between the probe 12 and the computing device 14. Additionally, as will be described in further detail herein, the cable 16 includes one or more conduits for exchanging heat from within the probe 12 to cooling air routed through the conduits.

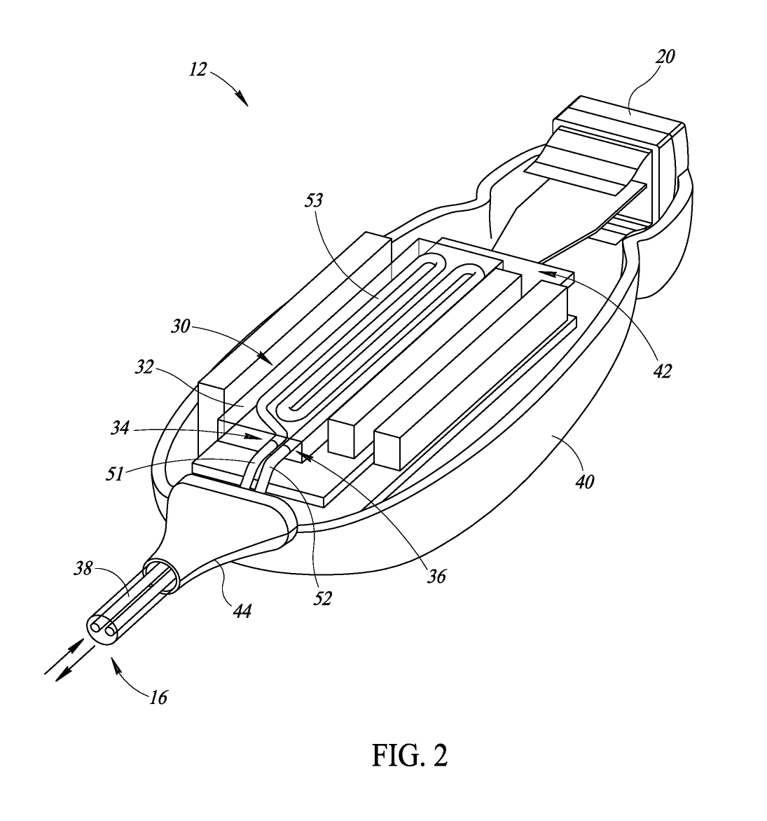

FIG. 2 is a cutaway view showing internal components of the transducer probe 12, in accordance with one or more embodiments. As can be seen from FIG. 2, the probe 12 includes various electrical or electronic components and circuitry located within a probe housing 40. In the cutaway view of FIG. 2, the upper portion of the probe housing 40 has been removed and thus is not shown. The probe housing 40 may be formed of a single piece (e.g., a single material that is molded surrounding the internal components) or may be formed of two or more pieces (e.g., upper and lower halves) which are bonded or otherwise attached to one another. The probe housing 40 may be a sealed housing, such that moisture, liquid or other fluids are prevented from entering the probe housing 40. In one or more embodiments, the probe housing 40 is sealed such that the probe 12 is liquid tight when submerged to a depth of at least one meter and is compliant with IPX7 of the IP Code (as published by the International Electrotechnical Commission).

The probe housing 40 surrounds internal electronic components and/or circuitry (shown generally at reference numeral 42) of the probe 12, including, for example, electronics such as driving circuitry, processing circuitry, oscillators, beamforming circuitry, filtering circuitry, and the like. The probe housing 40 may be formed to surround or at least partially surround portions of the probe 12, such as the imaging surface 20 (as shown in FIG. 2), and may be coupled to portions of the probe 12 (e.g., a cable sealing member 44) such that an interior of the probe 12 is sealed.

A heat exchanger 30 is provided within the interior of the probe 12 and includes a heat exchanger housing 32, an inlet port 34 and an outlet port 36. A fluid-tight tube or conduit 38 is routed into the heat exchanger housing 32 through the inlet port 34 and exits the heat exchanger 30 through the outlet port 36.

As shown in FIG. 2, the heat exchanger 30 may be a four-pass heat exchanger, which allows air or another fluid traveling through the conduit 38 to have a dwell time (i.e., a time from when a flow of air enters the heat exchanger housing 32 through the inlet port 34 until a time the flow of air exits the heat exchanger housing 32 through the outlet port 36) sufficient to absorb a suitable amount of heat from within the interior of the probe 12. It will be readily appreciated that the heat exchanger 30 may be any heat exchanger having a conduit 38 of suitable material and a suitable length for carrying a flow of air or any other cooling fluid.

The probe housing 40 may include an opening (not shown) through which electrical wires (e.g., for transmitting signals between the computing device 14 and the transducer probe 12) and the conduit 38 are routed into the probe 12, i.e., at the interface between the probe 12 and the cable 16. The cable sealing member 44 forms a fluid-tight seal between the cable 16 and the probe 12.

The heat exchanger housing 32 may be formed of a material having a high thermal conductivity, such as copper, aluminum, silver, gold or the like, including any alloys and composites thereof. Similarly, the conduit 38 may be formed of a high thermal conductivity material, such as copper, aluminum, silver, gold or the like, including any alloys and/or composites thereof.

In one or more embodiments, a first portion of the conduit 38 positioned within the heat exchanger housing 32 may be formed of a first material, and a second portion of the conduit 38 (i.e., the portion coupled to the heat exchanger housing 32 and provided through the cable 16) may be formed of a second material. The first portion of the conduit 38 may be formed of a material having a high thermal conductivity, while the second portion may be formed of any material, including, for example, a material having a low thermal conductivity (including, for example, polymers, plastics or the like). In alternative embodiments where the second portion of the conduit 38 is formed of high thermal conductivity material, the cable 16 may serve to help dissipate heat from the probe 12 to the surrounding environment, e.g., by convection through the outer casing of the cable 16.

In one or more embodiments, the conduit 38 may be formed of a single piece of material. Alternatively, the conduit 38 may include three or more separate portions which are mated to one another so as to form a single fluid-tight conduit. For example, as shown in FIG. 2, the conduit 38 may include an inlet tube 51, an outlet tube 52 and a heat exchanger tube 53. The inlet tube 51 may be mated or coupled to the inlet port 34 in a fluid-tight or otherwise sealed fashion. For example, the inlet port 34 may include a port that extends outwardly from the heat exchanger housing 32, and the inlet tube 51 may be positioned over and covering the port. The outlet tube 52 may similarly be mated or coupled to the outlet port 36 in a fluid-tight or otherwise sealed fashion. Once sealed, the inlet tube 51, outlet tube 52 and heat exchanger tube 53 form a single conduit 38 through which air or another cooling fluid may flow.

The conduit 38 may be a flexible conduit, which may be formed of any flexible material. In one or more embodiments, the inlet and outlet tubes 51, 52 may be formed of a flexible material, while the heat exchanger tube 53 may be formed of a rigid or semi-rigid material, such as copper tubing.

While the heat exchanger 30 is described herein as including a heat exchanger tube 53 and a heat exchanger housing 32, in one or more embodiments, the heat exchanger housing 32 itself forms or defines the heat exchanger tube 53. For example, in one or more embodiments, the heat exchanger housing 32 may be formed (e.g., machined) of one or more parts that, when assembled, define a fluid channel (i.e., the heat exchanger tube 53). In an embodiment, the heat exchanger housing 32 may include two halves, with each half having a portion (e.g., a half-circular portion, in cross-section) of a fluid channel being machined into it. The two halves may then be secured to one another, with the two portions of the fluid channel aligning such that a complete fluid channel is formed through the heat exchanger 30.

During normal operational use, the fluid pressures (e.g., from a flow of air) through the conduit 38 are generally low (e.g., in the range of about a thousand Pascals to a few thousand Pascals). The conduit 38 can thus have thin outer walls in order to maximize the inside diameter through which the cooling fluid will flow. A larger inside diameter allows for easier fluid flow (i.e., an increased flowrate at lower pressure as compared to a smaller inside diameter tube).

Air or any other cooling fluid may be pushed through the conduit 38 by a fluid pump 60 (FIG. 1) which may be located within the computing device 14. While the fluid pump 60 is shown in FIG. 1 as being located within the computing device 14, it will be readily appreciated that the fluid pump 60 may be positioned anywhere within the ultrasound device 10 and coupled to a cooling fluid inlet of the conduit 38 (e.g., at an inlet of the inlet tube 51), including, for example, within the connector 18, the cable 16 or the probe 12. The fluid pump 60 may be any fluid blower or pump device, and may preferably be a micro-pump or micro-blower, such as a miniature piezoelectric air pump.

During operation of the ultrasound device 10, heat is generated by the electronic components and circuitry 42 within the transducer probe 12 (e.g., the driving circuitry, processing circuitry, power source, transducer elements, etc.). Without cooling (such as provided, for example, by the heat exchanger 30 disclosed herein), the generated heat is passed via convection and/or radiation to an outer surface of the probe 12, such as the imaging surface 20. The temperature of the imaging surface 20 and/or an outer surface of the probe housing 40, without cooling, may thus rise to a level that is uncomfortable or unsafe to touch, or which may be beyond prescribed temperature limitations for ultrasound imaging devices.

However, in the ultrasound device 10 disclosed herein, the heat exchanger 30 actively removes heat from within the probe 12, which thus decreases the temperature of the outer surface of the probe housing 40 and the imaging surface 20 during operation of the ultrasound device 10 as compared with the temperature of such a device without a heat exchanger or heat transfer device as disclosed herein. Accordingly, the ultrasound device 10 increases an amount of heat that can be produced (and dissipated) by the probe 12, while still operating within safe or prescribed operational temperature limits.

In operation, air or any other cooling fluid is pumped through the conduit 38 by the fluid pump 60. The pumped fluid (e.g., air) flows into the heat exchanger 30 (e.g., through the inlet port 34) where it absorbs heat generated by the electronic components and circuitry 42 positioned in the interior of the probe 12 and carries the absorbed heat (e.g., the heated air) outside of the heat exchanger 30 (e.g., through the outlet port 36) and outside of the probe 12. The heat exchanger 30, conduit 38 and fluid pump 60 thus cool the probe 12 through forced convection.

The cable 16 may include one or more outlet vents 26 for venting the fluid (e.g., heated air) after it has absorbed heat from the probe 12 (e.g., by passing through the heat exchanger 30) and has exited the heat exchanger 30 and the probe 12. The outlet vents 26 may be coupled, for example, to the outlet tube 52 and may be located anywhere along the cable 16 or at the connector 18. In one or more embodiments, a vent may be provided within the computing device 14 and coupled to an outlet end of the outlet tube 52 for venting the heated fluid.

As described herein, the conduit 38 may carry air or any other cooling fluid for cooling the transducer probe 12. In one or more embodiments, the cooling fluid may be air or any other inert gas, which may provide certain advantages, such as being relatively easy to push through the conduit 38. Additionally, if the conduit 38 were to develop a leak, any leakage of air into the probe 12 would not cause damage or malfunctioning of the probe (e.g., via a short circuit) as may happen if another corrosive or conductive fluid were used as the cooling fluid.

The various embodiments described above can be combined and/or modified to provide further embodiments without departing from the scope of the present disclosure. For example, while the conduit 38 and heat exchanger 30 have been described herein as carrying cooling air, it will be readily appreciated that other fluids may be used in place of air as a coolant or heat transfer medium traveling through the conduit 38, including, for example, water or any other suitable cooling fluid.

In one or more embodiments, the ultrasound device 10 may include a closed cooling system. A fluid reservoir (containing any cooling fluid, including, for example, air, water or the like) may be provided, for example, in the computing device 14. The fluid pump 60 pumps and circulates the cooling fluid through the conduit 38, from the fluid reservoir to the heat exchanger 30 and back to the fluid reservoir. As the cooling fluid travels through the heat exchanger 30, it absorbs heat from within the ultrasound probe 12, which is then dissipated as the warmed fluid returns to the fluid reservoir (e.g., through the cable 16, the computing device 14 and/or a heat transfer device located in the computing device 14). In such embodiments, the fluid may be air or any other inert gas, as described above, or may be any liquid cooling fluid, such as water. A closed cooling system as described herein may eliminate problems such as dust, foreign materials, humid air or the like entering the cooling system.

In one or more embodiments, the computing device 14 may include a heat exchanger for removing heat from the fluid circulating in the conduit 38 (i.e., from the fluid that exits the heat exchanger 30 via the outlet tube 52 after absorbing heat from inside the probe 12). For example, in addition or as an alternative to vents being provided along the cable 16 or within the computing device 14, the outlet tube 52 may be coupled to a heat transfer device located within the computing device 14. The heat transfer device may be, for example, a heat dissipation element such as metallic fins that provide passive cooling (e.g., by convection and/or radiation), or a heat exchanger such as a thermoelectric cooler, thermal refrigeration unit or the like that provides active cooling.

Additionally, while the conduit 38 has been described herein as being provided within a cable 16 that also carries electrical wires (e.g., for transmitting signals between the computing device 14 and the probe 12), in one or more embodiments, the conduit 38 may be carried in a separate cable which may be inserted (and sealed) into the probe 12. As such, the present disclosure enables retrofitting conventional transducer probes, which may already include a single cable for carrying electrical wires, to include the heat transfer features and functionalities described herein. For example, a conventional ultrasound probe may be retrofitted by providing the conduit 38 through a separate cooling fluid cable that is coupled to the heat exchanger 30, which may be placed inside the transducer probe housing and then sealed.

While embodiments of the present disclosure are described as operatively removing heat from within the ultrasound probel2, it will be readily appreciated that embodiments provided herein may, additionally or alternatively, maintain a surface of the ultrasound probe 12 (e.g., the imaging surface 20, an outer surface of the probe 12, or the like) at an acceptable operational temperature, by removing heat from within the probe12, as described herein, by moving heat within the probe 12 away from the imaging surface 20 or other surface of the probe, or by removing heat from the surface of the probe 12 itself. In that regard, the heat exchanger 30 may be positioned anywhere within the ultrasound probe 12, or may be positioned outside of, and operatively coupled to, the ultrasound probe 12 (e.g., positioned adjacent to an outer surface of the probe 12 and operable to absorb heat from the outer surface of the probe 12).

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An ultrasound transducer probe, comprising:
an imaging surface including one or more transducer elements;
electronic circuitry including:
processing circuitry that controls transmission of an ultrasound signal from the one or more transducer elements, and
driving circuitry operatively coupled to the one or more transducer elements and the processing circuitry, the driving circuitry driving the transmission of the ultrasound signal from the one or more transducer elements in response to a control signal received from the processing circuitry;
a heat exchanger including a conduit that, in operation, conveys a flow of cooling fluid; and
a housing at least partially surrounding the electronic circuitry, the heat exchanger, and the imaging surface.

2. The ultrasound transducer probe of claim 1, wherein the heat exchanger includes an inlet port coupled to a first end of the conduit, and an outlet port coupled to a second end of the conduit, the ultrasound transducer probe further comprising:
an inlet conduit coupled to the inlet port of the heat exchanger; and
an outlet conduit coupled to the outlet port of the heat exchanger.

3. The ultrasound transducer probe of claim 2, further comprising a cable, the inlet and outlet conduits being at least partially routed through the cable.

4. The ultrasound transducer probe of claim 3, wherein the cable includes one or more outlet vents coupled to the outlet conduit.

5. An ultrasound system, comprising:
a transducer probe including:
an imaging surface including one or more transducer elements;
electronic circuitry coupled to the one or more transducer elements;
a heat exchanger including a conduit configured to convey a flow of fluid, the heat exchanger being configured to absorb heat generated in the transducer probe into the flow of fluid; and
a housing at least partially surrounding the electronic circuitry, the heat exchanger, and the imaging surface; and
a computing device operatively coupled to the transducer probe.

6. The ultrasound system of claim 5, wherein the heat exchanger includes an inlet port coupled to a first end of the conduit, and an outlet port coupled to a second end of the conduit, the transducer probe further including:
an inlet conduit coupled to the inlet port of the heat exchanger; and
an outlet conduit coupled to the outlet port of the heat exchanger.

7. The ultrasound system of claim 6, further comprising a cable, the inlet and outlet conduits being at least partially routed through the cable.

8. The ultrasound system of claim 7, wherein the cable includes one or more outlet vents coupled to the outlet conduit.

9. The ultrasound system of claim 7, wherein the computing device is operatively coupled to the transducer probe via the cable.

10. The ultrasound system of claim 9, further comprising:
a pump coupled to the inlet conduit and configured to provide the flow of cooling fluid to the conduit in the heat exchanger.

11. The ultrasound system of claim 10, wherein the cooling fluid is air, and the pump is a piezoelectric air pump.

12. The ultrasound system of claim 10, wherein the pump is positioned within the computing device.

13. The ultrasound system of claim 6, wherein the computing device includes a heat transfer device coupled to the outlet conduit, the heat transfer device being configured to cool the fluid.

14. The ultrasound system of claim 13, wherein the heat transfer device in the computing device includes a heat dissipation element configured to passively cool the fluid.

15. The ultrasound system of claim 13, wherein the heat transfer device in the computing device includes a heat exchanger configured to actively cool the fluid.

16. A method, comprising:
providing a heat exchanger within an ultrasound transducer probe, the heat exchanger including a heat exchanger conduit configured to convey a flow of fluid, the heat exchanger conduit including an inlet and an outlet;
coupling an inlet conduit to the inlet of the heat exchanger conduit;
coupling an outlet conduit to the outlet of the heat exchanger conduit; and
coupling a fluid pump to the inlet conduit, the fluid pump being configured to provide the flow of fluid by pumping a cooling fluid into the inlet conduit.

17. The method of claim 16, further comprising:
coupling a first end of a cable to the ultrasound transducer probe, the inlet and outlet conduits being at least partially routed through the cable.

18. The method of claim 17, wherein the cable includes one or more outlet vents in fluid communication with the outlet conduit.

19. The method of claim 16, wherein the cooling fluid is air, and the fluid pump is a piezoelectric air pump.

20. The ultrasound system of claim 17, wherein the fluid pump is located within a computing device, and wherein coupling the fluid pump to the inlet conduit includes coupling a second end of the cable to the computing device.
